(19) United States
(12) Patent Application Publication
(10) Pub. No.: US 2024/0139528 A1
(43) Pub. Date: May 2, 2024

(54) CONNECTOR FOR AN ELECTROMEDICAL ELECTRODE, A MATING CONNECTOR, ELECTROMEDICAL PULSE GENERATOR, ELECTROMEDICAL ELECTRODE, AND ELECTROMEDICAL PLUG CONNECTION

(71) Applicant: OSYPKA AG, Rheinfelden-Baden (DE)

(72) Inventors: Benjamin BURG, Rheinfelden (DE); Maik GR¿FE, Rheinfelden (DE); Thorsten GÖTTSCHE, Rheinfelden (DE)

(73) Assignee: OSYPKA AG, Rheinfelden/Baden (DE)

(21) Appl. No.: 18/279,913

(22) PCT Filed: Mar. 11, 2022
(86) PCT No.: PCT/EP2022/056299

§ 371 Date: Sep. 1, 2023

(30) Foreign Application Priority Data
Mar. 11, 2021 (DE) 102021105989.5

(51) Int. Cl. A61N 1/375; H01R 4/2437

(52) U.S. Cl. CPC A61N 1/3752; H01R 4/2437

(57) ABSTRACT

Improvements in the technical field of electromedical plug-in connections are provided via a connection plug (1) for an electromedical electrode (2), amongst other things. The electromedical electrode has at least one contact pole (3), which is coated with a non-conductive protective layer (4) which can be locally opened at least in part.

### CROSS-REFERENCE TO RELATED APPLICATIONS

The present application is a 371 National Phase of PCT/EP2022/056299, filed Mar. 11, 2022, which claims priority from German Patent Application No. 10 2021 105 989.5, filed Mar. 11, 2021.

### TECHNICAL FIELD

The invention relates to a connection plug for an electromechanical electrode having at least one contact pole, to a corresponding mating plug, to an electromedical pulse generator, to an electromedical electrode and to an electromechanical plug-in connection.

### BACKGROUND

Electromedical electrodes are used, for example, to emit stimulation pulses from an electromedical pulse generator to a target tissue, for example to muscle tissue and/or a nerve of a patient. Electromedical electrodes are also used to receive electrical pulses from a patient, for example electrical pulses from a muscle, in particular from a heart muscle, and to transmit these electrical pulses to a corresponding evaluation unit via the electrode.

Electromedical electrodes have, at their proximal end, a connection plug by way of which they can be connected to an electromedical pulse generator and/or a corresponding evaluation unit.

At least one contact pole is formed on the connection plug of electromedical electrodes of this kind. When handling electrodes of this kind, the at least one contact pole should not be touched in order to avoid accidentally coupling signals into the electromedical electrode. This is the case particularly when the electrode, by way of its distal end, is already implanted in the target tissue of the patient.

### SUMMARY

The object of the invention is to provide a connection plug, a corresponding mating plug, an electromedical electrode and an electromedical plug-in connection comprising a connection plug and a mating plug, the handling of which is simplified, in particular when the electrode is connected to a pulse generator or an evaluation unit, and which avoid signals being accidentally coupled in.

In order to achieve the object, a connection plug of the kind mentioned at the outset is initially proposed, the connection plug having one or more of the means and features disclosed herein that is directed at a connection plug of this kind. Therefore, in particular, a connection plug for an electromedical electrode having at least one contact pole is proposed in order to achieve the object, the connection plug being characterized in that the at least one contact pole is coated with a non-conductive protective layer which can be locally opened at least in part.

In this way, a connection plug is provided, the at least one contact pole of which is protected by a non-conductive protective layer against undesired interference signals being coupled in and is insulated by the non-conductive protective layer. The non-conductive protective layer can provide protection against contact with the at least one contact pole. The non-conductive protective layer, with which the contact pole is coated, can be locally opened at least in part immediately before the connection plug is connected to a corresponding mating plug in order to allow electrical contact between the contact pole and a corresponding mating contact pole of the mating plug.

The non-conductive protective layer can be coated, adhesively bonded, deposited or sprayed for example onto the at least one contact pole and/or generated on the at least one contact pole by the contact pole being clipped into an immersion bath containing a liquid, the non-conductive protective layer consisting of this liquid.

The non-conductive protective layer can be formed in such a way that it is automatically locally opened at least in part when the connection plug is connected to the mating plug. In this way, the separate removal of the protective layer, which removal may couple-in interference signals, before the connection plug is connected to the mating plug can be dispensed with.

Possible ways in which the non-conductive protective layer, with which the at least one contact pole of the connection plug is coated, can be locally opened at least in part automatically when the connection plug is connected to a corresponding mating plug are explained in more detail further below in the context of the mating plug according to the invention and also in the context of the specific exemplary embodiments of the invention.

In one embodiment of the connection plug, it is provided that the protective layer covers the at least one contact pole at least to such an extent that protection against contact with the contact pole is ensured. In a preferred embodiment of the connection plug, the protective layer covers the at least one contact pole completely. In this way, the at least one contact pole of the connection plug is reliably protected by the protective layer against control signals being accidentally coupled in.

The at least one contact pole can be electrically insulated by the protective layer.

It may be particularly expedient when the protective layer is a polymer layer which covers the at least one contact pole. Such a protective layer has a good electrically insulating effect and in addition can be comparatively easily applied to the at least one contact pole.

In one embodiment of the connection plug, it is provided that the at least one contact pole is ring-shaped. The at least one ring-shaped contact pole can be formed by a contact ring, which surrounds a longitudinal axis of the connection plug.

The at least one contact pole of the connection plug can be connectable or, in the use position of the connection plug, connected to an electrical conductor, for example to an electrical conductor of an electromedical electrode. The contact pole can function as a contact means which provides an interface for transmitting the signals transmitted to the contact pole via the abovementioned conductor to a corresponding mating contact pole.

In one embodiment of the connection plug, the at least one contact pole has, on a side that is averted from the protective layer and may be a rear side of the contact pole for example, a connecting interface for connection to an electrical conductor, in particular to an electrical conductor of an electromedical electrode. The contact pole can be connected to an electrical conductor via its connecting interface to an electrical conductor. After local opening of the protective layer, the contact pole which is connected for example at the rear side to the electrical conductor can be used for connection to a mating contact pole of a mating plug.

In one embodiment of the connection plug, the at least one contact pole has a round, preferably a circular, cross section. In another embodiment of the connection plug, the at least one contact pole has an angular, for example a rectangular, cross section. A contact pole with an angular, for example rectangular, cross section is suitable for fixing the connection plug in a manner secured against rotation on or in a mating plug, for example on or in a socket, which is used as a mating plug.

A mating plug having the means and features of the independent claim that is directed at a mating plug of this kind is also proposed in order to achieve the object. This mating plug may be a mating plug for the connection plug already explained above and/or for a connection plug as claimed in one of the claims that is directed at such a connection plug.

Therefore, in particular, a mating plug which has at least one mating contact pole and at least one means for locally opening a non-conductive protective layer of a contact pole of the connection plug at least in part in order to establish electrical contact between the at least one contact pole of the connection plug, which is coated with the non-conductive protective layer, and the at least one mating contact pole of the mating plug when the connection plug is connected to the mating plug is proposed in order to achieve the object.

In this way, a mating plug is provided, which is designed to locally open the non-conductive protective layer, with which the at least one contact pole of the connection plug is covered, at least in part when the plug-in connection between the connection plug and the mating plug is established, in order to establish an electrically conductive connection between the contact pole of the connection plug and the mating contact pole of the mating plug. In this way, separate handling for removing an electrical insulation before connecting the connection plug to the mating plug is avoided. This can considerably simplify the handling of an electromedical electrode which is provided with a connection plug and a mating plug of these kinds and additionally increase safety for a patient to be treated using such an electromedical electrode.

The at least one means, which is designed for locally opening the non-conductive protective layer of the contact pole of the connection plug at least in part, can be a cutting edge for example. The cutting edge can be arranged and/or formed on the mating plug and/or associated with the at least one mating contact pole.

The mating plug preferably has, for each mating contact pole, in each case at least one means of this kind for locally opening a non-conductive protective layer on a contact pole of a connection plug at least in part.

In one embodiment of the mating plug, the at least one means for locally opening the non-conductive protective layer at least in part is an annular cutting edge. The annular cutting edge can be formed on a fastening screw of the mating plug for the connection plug.

In one embodiment of the mating plug, it is provided that the mating plug has at least one insulation-displacement contact on which a mating contact pole and at least one means for locally opening a non-conductive protective layer of a contact pole of the connection plug at least in part are formed or arranged.

In this embodiment of the mating plug, it is particularly conveniently possible to establish the electrical connection between the at least one contact pole of the connection plug and the at least one mating contact pole of the mating plug automatically when the mating plug is connected to the connection plug.

It is particularly advantageous when the mating plug has a number of mating contact poles and/or means for locally opening the protective layer at least in part, the number of mating contact poles corresponding to a number of contact poles of the connection plug. In this way, it is possible to establish an electrical connection between all of the contact poles of the connection plug and mating contact poles of the mating plug automatically when the connection plug is connected to the mating plug.

In one embodiment of the mating plug, at least two, in particular a plurality of, mating contact poles which are, for example, axially spaced apart from each other are provided.

The mating plug can have a pull-out prevention means for a connection plug which is connected to the mating plug. In this way, the connection plug which is connected to the mating plug, in its contact position, can be reliably fixed to the mating plug. Unintentional releasing of the plug-in connection can be prevented by the pull-out prevention means.

The at least one means, which is provided for locally opening the protective layer of a contact pole of a connection plug, can serve as a pull-out prevention means for the mating plug for the connection plug which is connected to the mating plug. The pull-out prevention means can be provided, for example, by way of the means, for example the cutting edge, cutting into the coating and, similarly to a barb, in this way fixing the connection plug to the mating plug. It may be advantageous when the at least one means is oriented transversely and/or obliquely in relation to a pull-out direction, which is defined by the mating plug, of a connection plug and preferably against this pull-out direction. When the connection plug is pulled in the pull-out direction, a tensile force which is transmitted to the at least one means can be deflected due to the orientation of the means in such a way that the means is pressed against the connection plug, in particular against the contact pole of the connection plug, with which the means is associated, and in this way can prevent the connection plug from being released from the mating plug.

The at least one means, for example the cutting edge already mentioned above, can be arranged or formed on a support, for example on a sheet-metal strip. The at least one mating contact pole can be connectable or connected to an electrical conductor. In connection with the pull-out prevention means mentioned above, it may be advantageous when the support is oriented obliquely in relation to the pull-out direction and/or against the pull-out direction, which the mating plug for the connection plug can define. The support, which is oriented obliquely in relation to the pull-out direction, can span an acute angle with the pull-out direction. In order to provide pull-out prevention, the at least one means, in particular the cutting edge, can run in a plane which is oriented transversely or at a right angle to the pull-out direction and therefore can be oriented transversely or at a right angle in relation to the pull-out direction.

The mating plug can be in the form of a plug socket. The pull-out direction mentioned above can be oriented in the direction of the profile of a longitudinal axis of the plug socket. The mating plug, in particular if it is in the form of a plug socket, can be part of an electromedical pulse generator, for example a pacemaker.

An electromedical pulse generator, for example a pacemaker, having at least one mating plug as claimed in one of the claims that are directed at such a mating plug and/or having at least one connection plug as claimed in one of the claims that are directed at such a connection plug is also proposed in order to achieve the object.

An electromedical electrode having a connection plug and/or a mating plug as claimed in one of the preceding claims is also proposed in order to achieve the object.

Furthermore, an electromedical plug-in connection which has a connection plug and a mating plug in each case as claimed in one of the preceding claims is also proposed in order to achieve the object.

### BRIEF DESCRIPTION OF THE DRAWINGS

The invention will be explained in more detail below on the basis of exemplary embodiments, but is not restricted to these exemplary embodiments. Further exemplary embodiments can be gathered from combining the features of individual or multiple claims with one another and/or combining individual or multiple features of the exemplary embodiments.

Specifically:

FIG. 1 shows a proximal portion of an electromedical electrode having a connection plug on which a total of five contact poles which are in the form of ring contacts are formed,

FIG. 2 shows the electromedical electrode, illustrated in FIG. 1, with its connection plug, wherein all the contact poles of the connection plug are coated with a non-conductive protective layer here,

FIG. 3 shows a perspective illustration of an electromedical plug-in connection comprising a connection plug, as illustrated in FIGS. 1 and 2, and a mating plug which is designed to correspond to the connection plug and has a total of five mating contact poles with means for locally opening the non-conductive protective layer, which covers the contact poles of the connection plug, at least in part,

FIG. 4 shows a perspective view of a mating contact of a further mating plug which has, as means for fixing a connection plug, as is shown in FIGS. 1 to 3, a fastening screw and, as means for locally opening the non-conductive protective layer of the connection plug at least in part, an annular cutting edge at the screw-in end of the fastening screw,

FIG. 5 shows an exploded illustration of the mating contact shown in FIG. 4,

FIG. 6 shows a perspective illustration of parts of an electromedical plug-in connection which comprises a connection plug and a mating plug, wherein contact poles of the connection plug are shown in a connecting position on mating contact poles of the mating plug and each contact pole of the connection plug is connected to an electrical conductor,

FIG. 7 shows a further perspective illustration of the parts, shown in FIG. 6, of the plug-in connection for illustrating a pull-out prevention means which is provided by the mating plug for the connection plug, and

FIG. 8 shows a perspective illustration of a further plug-in connection comprising a mating plug and a connection plug which is connected to the mating plug,

FIG. 9 shows a perspective illustration of a further embodiment of a connection plug having contact poles which are rectangular in cross section and by which the connection plug can be fixed in a manner secured against rotation, for example, to a mating plug shown in the previous Figures, and

FIG. 10 shows a schematic illustration of an electromedical pulse generator having an electromedical electrode which is connected to the pulse generator via a plug-in connection according to the invention.

### DETAILED DESCRIPTION

In the following description of various embodiments of the invention, elements which correspond in terms of their function are provided with corresponding reference numerals even in the case of a different design or form.

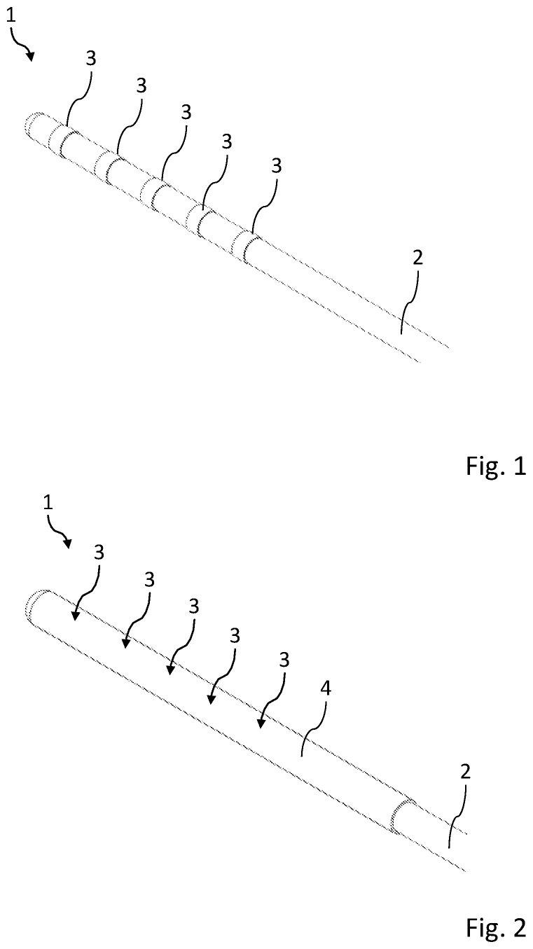

FIGS. 1 to 3 and FIGS. 6 to 10 show various embodiments of connection plugs designated 1 overall.

FIGS. 1 to 3 show a connection plug, designated 1 overall, which is arranged at one connection end, for example a distal end from the perspective of a patient, of an electromedical electrode 2. The connection plug 1 comprises a total of five contact poles 3. According to FIG. 2, each of the contact poles 3 is coated with a non-conductive protective layer 4 which can be locally opened at least in part in order to establish electrical contact with the contact poles 3. The connection plugs 1 shown in the other Figures are also constructed in accordance with this concept.

The protective layer 4 covers the contact poles 3 completely. The contact poles 3 of the connection plug 1 are electrically insulated by the protective layer 4, provided that the protective layer 4 has not yet been locally opened at least in part in the region of the contact poles 3. This prevents contact accidentally being made with the contact poles 3. The protective layer 4, which covers the contact poles 3, is a polymer layer. The protective layer 4 therefore functions as protection against contact with the contact poles 3.

The contact poles 3 of the connection plug 1 are ring-shaped contact poles which are formed by contact rings.

The contact poles 3 of the connection plug 1 are axially spaced apart from each other with respect to a longitudinal axis of the connection plug 1 and are uniformly distributed in one region.

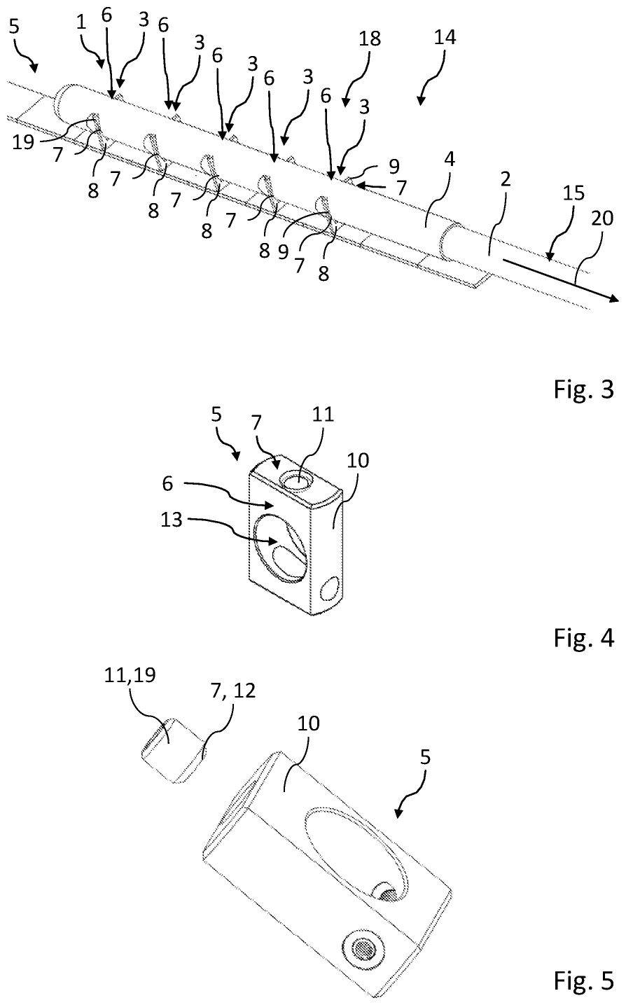

FIG. 3 shows a mating plug 5 which fits with the connection plug 1. The mating plug 5 has a total of five mating contact poles 6 which fit with the contact poles 3 of the connection plug 1. At least one means 7 for locally opening the non-conductive protective layer 4 of the contact poles 3 of the connection plug 1 at least in part is associated with each of the mating contact poles 6.

FIG. 3 shows that the mating plug 5, due to the means 7 for locally opening the protective layer 4 on the contact poles 3 of the connection plug 1 at least in part, is designed to establish electrical contact between the contact poles 3 of the connection plug 1 and the mating contact poles 6 of the mating plug 5 when the connection plug 1 is connected to the mating plug 5.

The means 7 for locally opening the protective layer 4 on the contact poles 3 of the connection plug 1 at least in part are cutting edges. In the exemplary embodiment of the mating plug 5 shown in FIG. 3, the mating plug has a total of five insulation-displacement contacts 8. In each case one mating contact pole 6 and at least one means 7 for locally opening the non-conductive protective layer 4 of the contact poles 3 of the connection plug 1 at least in part are formed or arranged on each of the insulation-displacement contacts 8.

FIG. 3 shows how the means 7, which are in the form of cutting edges, on the insulation-displacement contacts 8 are cut into the protective layer 4, which covers the contact poles 3 of the connection plug 1.

The insulation-displacement contacts 8 each have a pair of arms 9 on which the means 7 in the form of cutting edges are formed. The arms 9 of an insulation-displacement contact 8 are at a distance from each other here such that the means 7, here the cutting edges, reach the position shown in FIG. 3 on the mating plug 5 when the connection plug 1 is pressed in and in so doing cut through the non-conductive protective layer 4. In the process, the means 7 come into contact with the contact poles 3, as a result of which the desired electrical contact between the mating contact poles 6 and the contact poles 3 of the connection plug 1 is ultimately established.

FIGS. 4 and 5 show a mating contact 10 of a further mating plug 5 which is likewise designed to correspond to the connection plug 1 of FIGS. 1 to 3. This mating contact 10 also has a mating contact pole 6 and a means 7 for locally opening the protective layer 4, which covers the contact poles 3 of the connection plug 1, of the connection plug 1 at least in part.

The mating contact 10 is provided with a fastening screw in the form of a grub screw 11 on which the mating contact pole 6 and the means 7 for locally opening the protective layer 4 at least in part are designed in the form of an annular cutting edge 12.

In order to connect the connection plug 1 to the mating plug 5 which is provided with the mating contact 10, the connection plug 1 is pushed through an opening 13, shown in FIGS. 4 and 5, of the mating plug 10 until the connection plug 1, by way of one of its contact poles 3, enters the region of the mating contact pole 6 which is formed on the mating contact 10.

In this position, the fastening screw 11 is firmly screwed against the contact pole 3 located in the opening 13, wherein the annular cutting edge 12 of the fastening screw 11 cuts through the protective layer 4 until it comes into direct contact with the contact pole 3 of the connection plug 1.

Using the fastening screw 11, the connection plug 1 is therefore not only fixed to the mating contact 10 and as a result at least indirectly also to the mating plug 5, but also locally opens the protective layer 4, which covers the contact pole 3, at least in part in such a way that electrical contact is established between the mating contact pole 6 and the contact pole 3 of the connection plug 1.

FIG. 3 shows that the mating plug 5 has a number of mating contact poles 6 and means 7 for locally opening the protective layer 4 at least in part, the number of mating contact poles corresponding to the number of contact poles 3 of the connection plug 1.

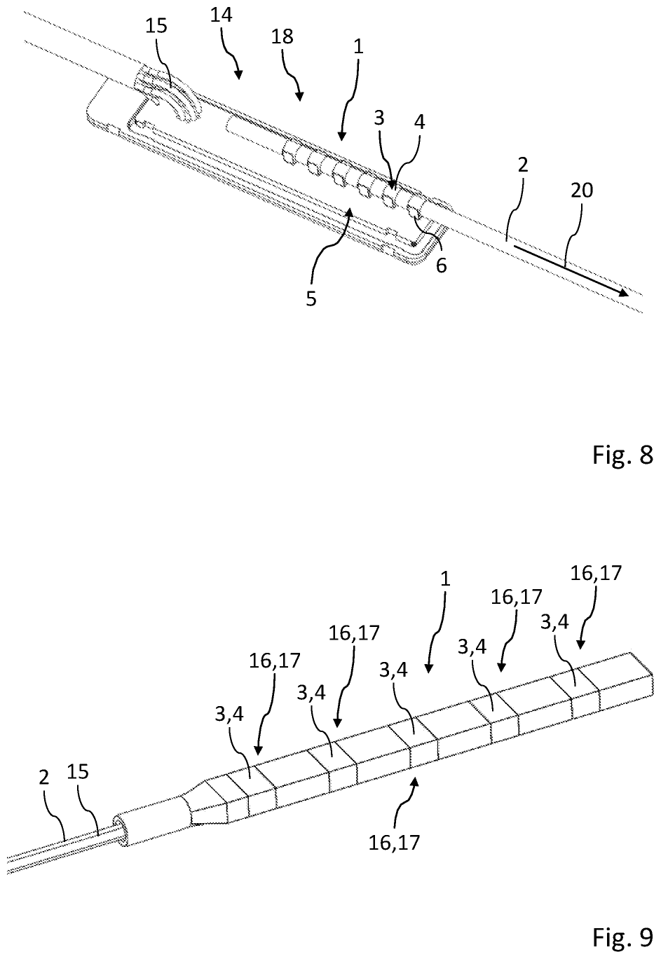

According to FIG. 3, the mating plug 5 has a total of five mating contact poles 6 which are axially spaced apart from each other. The connection plug 1 and the corresponding mating plug 5 together form an electromedical plug-in connection 14. Such a plug-in connection 14 is also shown in FIG. 8.

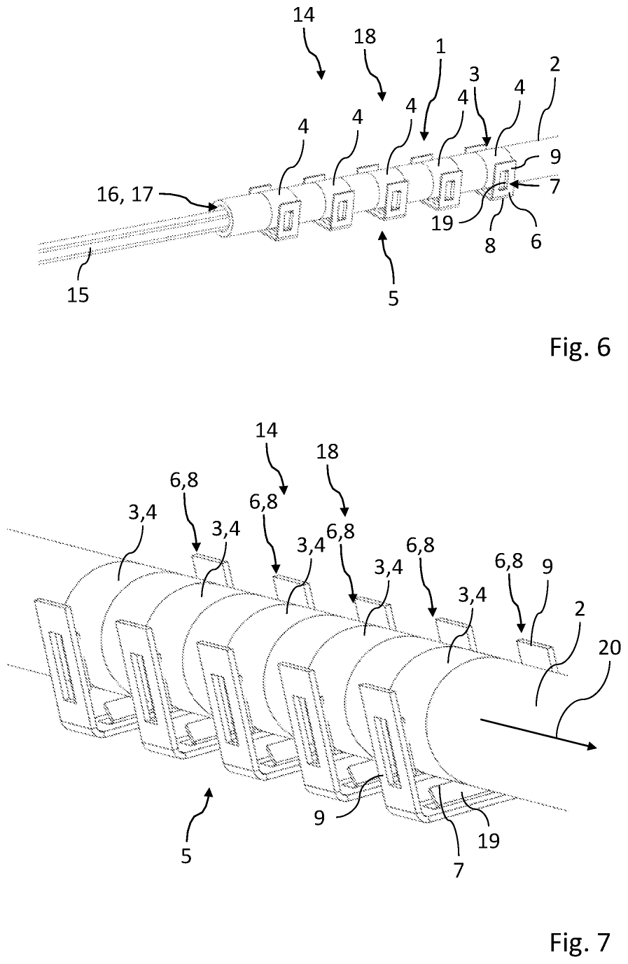

The previous embodiments with respect to the connection plug 1 shown in FIGS. 1-3 can be transferred to the connection plug 1 shown in FIGS. 6-10.

FIG. 6 shows that the contact poles 3 of the connection plug 1 shown there are each connected to an electrical conductor 15 and are each covered by an electrically non-conductive protective layer 4. The protective layer 4 on the contact poles 3 functions as protection against contact and prevents pulses from being unintentionally introduced into the conductors 15, which are connected to the contact poles 3, of an electromedical electrode 2. The connection plug 1 is arranged at a connection end of the electrode 2. The conductors 15 of the electrode 2 are connected to the contact poles 3 of the connection plug 1 and themselves are in the form of stranded wires which are provided with an insulation.

The contact poles 3, on their side 16 which is averted from the protective layer 4, each have a connecting interface 17 for connection to the electrical conductors 15 of the electromedical electrode 2. For example, the contact poles 3 shown in FIG. 6 are connected to the electrical conductors 15 via their connecting interfaces 17 on the side 16 that is averted from the protective layer 4. At the front side, the contact poles 3 are covered by the protective layer 4 already explained above. In this exemplary embodiment of the connection plug 1, the protective layer 4 can also be locally opened using the means 7 of the mating plug 5 in order to establish electrical contact between the contact poles 3 of the connection plug 1 and the corresponding mating contact poles 6 of the mating plug 5. This is done automatically when the connection plug 1 is moved to its connection position, shown in FIGS. 6 and 7, on the mating plug 5.

The connection plugs shown in FIGS. 1-3 and 6-8 have contact poles 3 which are formed not only in a ring shape but furthermore also with a circular cross section. These contact poles 3 have a cylindrical outer contour.

The connection plug 1 shown in FIG. 9, which likewise has a total of five contact poles 3 which are covered on the outer side by an insulating protective layer 4, is of rectangular cross section and accordingly has contact poles 3 of rectangular cross section. The design of the contact poles 3 with a rectangular cross section allows the connection plug 1 to be secured against rotation when it is in the connection position with a corresponding mating plug 5. The connection plug 1 shown in FIG. 9, instead of the connection plug 1 shown in FIG. 6 for example, can be connected to the mating contact poles 6 of the mating plug 5 shown in FIG. 6.

The mating contact poles 6 of the mating plug 5 shown in FIGS. 6, 7 and 8 are formed on the arms 9 of the U-shaped insulation-displacement contacts 8. Each insulation-displacement contact 8 has in each case three means 7, specifically cutting edges, for locally opening the protective layer 4 on the contact poles 3. In this way, it is possible to insert the connection plugs 1 shown in the Figures between the arms 9 of the insulation-displacement contacts 8, as a result of which the means 7, specifically the cutting edges of the insulation-displacement contacts 8, cut into the protective layer 4 on the contact poles 3, at least locally open the protective layer 4 here and establish electrical contact with the contact poles 3.

The mating plugs 5 shown in FIGS. 3-10 have a pull-out prevention means 18 for a connection plug 1 which is connected to the respective mating plug 5.

Here, the means 7, specifically the cutting edges of the mating plug 5 already mentioned above, can provide a pull-out prevention means 18 for the mating plug 5 for a connection plug 1 which is connected to the mating plug 5.

In the mating plug 5 shown in FIGS. 6-8, the means 7, specifically the cutting edges, are each arranged or formed at a free end of a support 19, specifically a sheet-metal strip. The supports 19 are bent away from the arms 9 of the insulation-displacement contacts 8 of the mating plug 5 shown in FIGS. 6-8. In the mating plug 5 shown in FIG. 6, the means 7 and the supports 19 are oriented in the direction of a pull-out direction 20 which is defined by the mating plug 5 and its insulation-displacement contacts 8.

FIG. 7 shows the mating plug 5 in an orientation rotated through 180° relative to the connection plug 1 in comparison to FIG. 6. The supports 19 and the means 7, specifically the cutting edges of the insulation-displacement contacts 8, are as can be seen here oriented obliquely in relation to and against the pull-out direction 20 of the connection plug 1 out of the mating plug 5 and span an acute angle with the pull-out direction 20. In addition, the means 7, specifically the cutting edges, also run in a plane which is oriented at a right angle to the pull-out direction 20. When the connection plug 1 is pulled in the pull-out direction 20, a tensile force which is transmitted to the means 7 in so doing can be deflected, on account of the orientation of the means 7, in such a way that the means 7 are pressed against the connection plug 1, specifically against its contact poles 3, serve as pull-out prevention means 18 and prevent the connection plug 1 from being released from the mating plug 5. The means 7 which are provided on the supports 19 function here as barbs which hold the connection plug 1 in the connection position on the mating plug 5.

In the embodiments of the mating plug 5 which are shown in FIGS. 3 to 5, the arms 9 of the insulation-displacement contacts 8 or the grub screw 11 function as supports 19 for the means 7. Each of the insulation-displacement contacts 8 shown in FIGS. 6-8 have three means 7 in the form of cutting edges. Two of the means 7 are arranged on mutually facing sides of the arms 9 of the insulation-displacement contacts 8. A third means 7 is arranged on a connecting portion of the respective insulation-displacement contact 8 which connects the two arms 9 to each other.

FIG. 8 of an electromedical plug-in connection 14 shows that the contact poles 6 of the mating plug 5 are likewise connected to at least one electrical conductor 15 in each case.

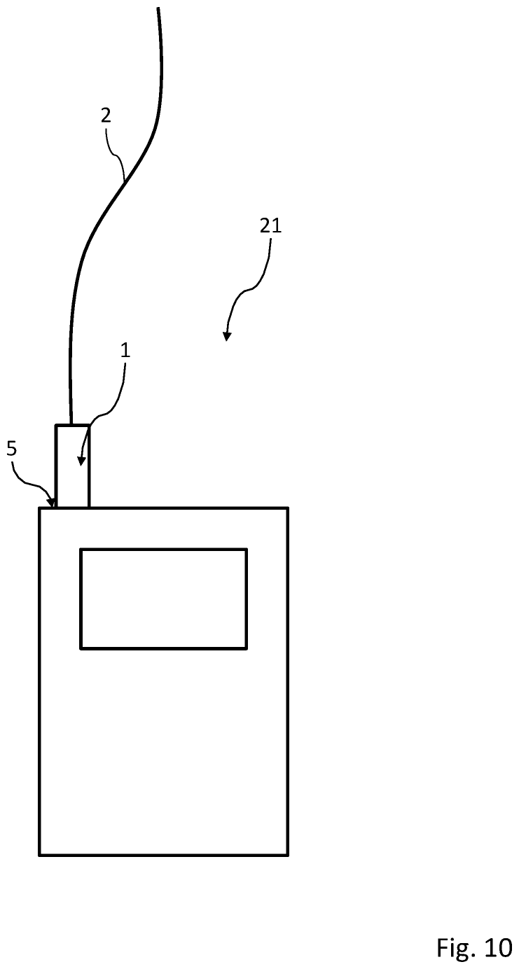

FIG. 10 shows an electromedical pulse generator 21 which has a mating plug 5 according to the invention in the form of a plug socket. An electromedical electrode 2 which is equipped with a suitable connection plug 1 can be connected to the electromedical pulse generator 21 via the mating plug 5. The electromedical pulse generator 21 can be, for example, a pacemaker, e.g. a cardiac pacemaker. The electromedical electrode 2 shown in FIG. 10 has a connection plug 1 at one of its ends that can be referred to as the distal end of the electrode 2 from the perspective of the patient and as the proximal end of the electrode 2 from the perspective of the pulse generator 21. The contact poles 3 of the connection plug 1 are each connected to an electrical conductor 15 of the electrode 2 here.

The invention concerns improvements in the technical field of electromedical plug-in connections. A connection plug 1 for an electromedical electrode 2, amongst other things, is proposed for this purpose, the electromedical electrode having at least one contact pole 3, which is coated with a non-conductive protective layer 4 which can be locally opened at least in part.

### LIST OF REFERENCE SIGNS

1 Connection plug
2 Electromedical electrode
3 Contact pole
4 Protective layer
5 Mating plug
6 Mating contact pole
7 Means for locally opening 4 at least in part
8 Insulation-displacement contact
9 Arms of 8
10 Mating contact 9
11 Fastening screw/grub screw
12 Annular cutting edge
13 Opening of 10
14 Electromedical plug-in connection
15 Electrical conductor
16 Side of 3 that is averted from the protective layer 4
17 Connecting interface
18 Pull-out prevention means
19 Support
20 Pull-out direction
21 Electromedical pulse generator

1. A connection plug (1) for an electromedical electrode (2), the connection plug comprising:
at least one contact pole (3) that is coated with a non-conductive protective layer (4) which is locally opened at least in part.

2. The connection plug (1) as claimed in claim 1, wherein at least one of a) the non-conductive protective layer (4) provides protection against contact with the at least one contact pole (3), the protective layer (4) covers the at least one contact pole (3) at least to such an extent that protection against contact with the at least one contact pole (3) is ensured, or the protective layer (4) covers the at least one contact pole (3) completely.

3. The connection plug (1) as claimed in claim 1, wherein at least one of a) the at least one contact pole (3) is electrically insulated by the protective layer (4) or b) the protective layer (4) is a polymer layer which covers the at least one contact pole (3).

4. The connection plug (1) as claimed in claim 1, wherein the at least one contact pole (3) is ring-shaped.

5. The connection plug (1) as claimed in claim 1, wherein the connection plug (1) has at least two contact poles (3) which are axially spaced apart from each other.

6. The connection plug (1) as claimed in claim 1, wherein the at least one contact pole (3) is connectable or connected to an electrical conductor (15).

7. The connection plug (1) as claimed in claim 1, wherein the at least one contact pole (3) has, on a side (16) that is averted from the protective layer (4), a connecting interface (17) for connection to an electrical conductor (15).

8. The connection plug (1) as claimed in claim 1, wherein the at least one contact pole (3) has a round or an angular cross section.

9. A mating plug (5), comprising:
at least one mating contact pole (6); and
at least one means (7) for locally opening a non-conductive protective layer (4) of a contact pole (3) of the connection plug (1) at least in part in order to establish electrical contact between the at least one contact pole (3) of the connection plug (1) and the at least one mating contact pole (6) of the mating plug (5) when the connection plug (1) is connected to the mating plug (5).

10. The mating plug (5) as claimed in claim 9, wherein the at least one means (7) comprises a cutting edge.

11. The mating plug (5) as claimed in claim 9, wherein the mating plug (5) has at least one insulation-displacement contact (8) on which a mating contact pole (6) and the at least one means (7) for locally opening the non-conductive protective layer (4) of the at least one contact pole (3) of the connection plug (1) at least in part are formed or arranged.

12. The mating plug (5) as claimed in claim 9, wherein the at least one means (7) comprises an annular cutting edge (12) formed on a fastening screw (11) of the mating plug (5) for the connection plug (1).

13. The mating plug (5) as claimed in claim 9, wherein the mating plug (5) has at least one of a number of the mating contact poles (6) or a plurality of the means (7) for locally opening the protective layer (4) at least in part, the number of mating contact poles corresponding to the number of the contact poles (3) of the connection plug (1).

14. The mating plug (5) as claimed in claim 9, wherein the mating plug (5) has at least two of the mating contact poles (6) which are axially spaced apart from each other.

15. The mating plug (5) as claimed in claim 9, wherein the mating plug (5) has a pull-out prevention means (18) for a connection plug (1) which is connected to the mating plug (5).

16. The mating plug (5) as claimed in claim 15, wherein the at least one means (7) provides the pull-out prevention means (18) for the mating plug (5) for the connection plug (1) which is connected to the mating plug (5), and the at least one means (7) is oriented transversely or at a right angle to a pull-out direction (20) which is defined by the mating plug (5).

17. The mating plug (5) as claimed in claim 9, wherein the at least one means (7) is arranged or formed on a support (19).

18. The mating plug (5) as claimed in claim 9, wherein the at least one mating contact pole (6) is connectable or connected to an electrical conductor (15).

19. An electromedical pulse generator (21), comprising at least one mating plug (5) as claimed in claim 9 and a connection plug (1) with at least one contact pole (3) that is coated with a non-conductive protective layer (4) which is locally opened at least in part by the connection to the at least one mating plug (5).

20. An electromedical electrode (2) comprising at least one of a) a connection plug (1) with at least one contact pole (3) that is coated with a non-conductive protective layer (4) which is locally opened at least in part by the connection to at least one mating plug (5) or b) a mating plug (5) having at least one mating contact pole (6), and at least one means (7) for locally opening a non-conductive protective layer (4) of a contact pole (3) of the connection plug (1) at least in part in order to establish electrical contact between the at least one contact pole (3) of the connection plug (1) and the at least one mating contact pole (6) of the mating plug (5) when the connection plug (1) is connected to the mating plug (5).

21. The electromedical electrode (2) as claimed in claim 20, wherein at least one of the at least one contact pole (3) of the connection plug (1) or the at least one mating contact pole (6) of the mating plug (5) is connected to an electrical conductor (15).

22. An electromedical plug-in connection (14) comprising a connection plug (1) with at least one contact pole (3) that is coated with a non-conductive protective layer (4) which is locally opened at least in part by the connection to at least one mating plug (5) and a mating plug (5) having at least one mating contact pole (6), and at least one means (7) for locally opening a non-conductive protective layer (4) of a contact pole (3) of the connection plug (1) at least in part in order to establish electrical contact between the at least one contact pole (3) of the connection plug (1) and the at least one mating contact pole (6) of the mating plug (5) when the connection plug (1) is connected to the mating plug (5).
